(19) United States
(12) Patent Application Publication
(10) Pub. No.: US 2010/0004170 A1
(43) Pub. Date: Jan. 7, 2010

(54) PROTEIN-CONTAINING SUBSTANCE WITH INCREASED THERMAL STABILITY

(75) Inventors: Bruno Winter, Stuttgart (DE); Stephen Cole, Staffordshire (DE)

Correspondence Address: EDWARDS ANGELL PALMER & DODGE LLP, P.O. BOX 55874, BOSTON, MA, 02205, US

(73) Assignee: AB Enzymes GmbH, Darmstadt (DE)

(21) Appl. No.: 12/312,357

(22) PCT Filed: Nov. 2, 2007
(86) PCT No.: PCT/EP2007/009523

§ 371 Date: Jul. 14, 2009

(30) Foreign Application Priority Data
Nov. 10, 2006 (DE) 10 2006 053 071.3

(51) Int. Cl. A61K 38/47; A61K 38/16; A61K 38/43; A61K 38/46; B05D 3/00; A61P 43/00; A23K 1/16; A23L 1/20; A23K 1/14

(52) U.S. Cl. 514/12; 424/94.1; 424/94.6; 424/94.61; 427/384; 426/93; 426/289

(57) ABSTRACT

The invention relates to an agent with increased thermal stability comprising a core and a coating layer characterized in that the core comprises at least one biologically active protein and that the coating layer comprises a micronized product from leguminous plants, a method for the production of this agent as well as the use of a micronized product from leguminous plants to increase the thermal stability of biologically active proteins in methods for the production of a granulated product as well as in methods for the production of animal feed, food and pharmaceuticals.

The invention relates to a protein-containing agent with increased thermal stability comprising a core with at least one biologically active protein and a coating layer that comprises a micronized product from leguminous plants. The invention further relates to a method for the production of a protein-containing agent with increased thermal stability as well as the use of a micronized product from leguminous plants to increase the thermal stability of biologically active proteins in methods for the production of granulated and/or pelletized products, keeping up the high biological availability of the protein-containing agent.

Biologically active proteins and particularly enzymes are usually used as dry granulated preparations. Protein-containing products are easy to manage and to dose in this form. Numerous technologies are known for the production of such granulated products. For example, in fluidized bed dryers (fluid bed agglomerators) particles are dispersed in the air current over a porous carrier and form agglomerates, which are subsequently optionally coated and finally dried. In mixing agglomerators such as, e.g., plow blade mixers, the formation of agglomerates or granulates happens by particle collision, whereby dense or less dense agglomerates may be formed corresponding to the shear forces. These agglomerates may be subsequently dried to a certain residual humidity. Moreover, extruded or pelletized products in which a paste containing the protein is pressed to pellets or is extruded through a little opening under pressure and then cut in particles, which are subsequently dried, are also often used. In all these methods more or less drastic conditions as regards shear forces and temperature are used. Proteins, particularly enzymes, that are used in the field of animal feed production may be exposed to such loads in the course of their processing, whereby the protein or the enzyme may be limited in its function. For example, in the incorporation of a pulverized or granulated enzyme into animal feed pellets an inactivation of the enzyme may take place by contact with the inhibitors in the feed and/or by the increased temperature and humidity in the steam flow in the conditioning step of the pelletizing process of the animal feed.

There have already been experiments as to avoid the inactivation and particularly the thermal inactivation of biologically active proteins in such manufacturing methods. For example, enzymes were not covalently immobilized on relatively large particles (e.g., starch-containing particles from cassava starch, sago starch, rice starch, wheat starch, sorghum starch or barley starch; cf. WO 97/39116 of silica particles (cf. U.S. Pat. No. 5,318,903; WO 94/26883) or vegetable flours were used as carriers (cf. U.S. Pat. No. 4,106,991). Moreover, the water content near by the substance was reduced, e.g., by sodium sulfate (cf. WO 97/39116, WO 92/12645, WO 01/04279), and/or a water-resistant and steam-resistant barrier, i.e., a coating, was conducted to protect the biologically sensitive substances at an increased water content from high temperatures and their effects alone or in combination with water or water vapor (cf. WO 97/39116, U.S. Pat. No. 6,610,519, WO 00/01793, WO 92/12645, WO 96/16151, WO 01/04279). Furthermore, components that are to absorb preferably thermal and mechanical energy were specifically added to enzyme granulates. The use of lactic acid to stabilize phytase is disclosed in WO 00/20569.

In the case of enzymes enzyme stabilizers were also added, for example, trehalose and zinc sulfate, corn steep water, phytic acid and inositol monophosphate (in the case of phytase). The addition of corn steep water, which also comprises phytic acid and inositol phosphates, is, e.g., disclosed in WO 00/20569. The addition of the specific inorganic salts zinc sulfate and magnesium sulfate in particular is disclosed in U.S. Pat. No. 5,972,669, U.S. Pat. No. 5,827,709 as well as EP 0 758 018.

The use of trehalose, zinc sulfate and polyethylene glycol for the coating is disclosed in WO 98/55599.

However, these methods have the disadvantage that additions such as zinc sulfate, a trace element, in high amounts (>15%; cf. U.S. Pat. No. 5,827,709) are not applicable to all feed rations of animals. Coatings made of hydrophobic materials such as wax, fat, etc. reduce the biological availability of the enzyme in that the enzyme is only very lately released in the stomach and, thus, must be dosed in high amounts to be able to show efficacy, e.g., to be able to act on the pig within the timely restricted retention time of about 4 h. Moreover, many of these compositions are only specifically optimized and even effective for certain enzymes such as, e.g., phytase. Other additions such as kaolin, zeoliths and silica are insoluble compounds that cannot be biologically degraded, which strongly limits their use. The spraying on carriers or the absorption by swellable polysaccharides limits the applicable amount of protein and the throughput in the production, since it would otherwise result in clumping during the spray process. The enzyme activity to be maximally obtained in thus produced enzyme particles is also limited by the ratio of carrier to applicable enzyme.

Thus, there is a need for a method to increase the thermal stability of biologically active proteins. In particular, the thermal stability of biologically active proteins for the use in methods for the production of agents with defined and/or small grain size, preferably for the use in methods for the production of, e.g., pelletized products, is to be increased. The thermal stability, that is the recovery of the used enzyme activity after thermal loads, in enzyme preparations is to be particularly increased. The production method is to be easily and reliably applicable. Furthermore, the method is to result in granulated products that are not toxic and harmless for human and animal consumption. The method is to be universally applicable for any agglomeration products, granulation products and extrusion products and is to guarantee a sufficient temperature stability of the enzymes in the application of the thus produced products even at high process temperatures. Moreover, by the substances used in this method, no other negative interaction with the biologically active proteins is to take place. In addition, the method is to be environmentally friendly, i.e., agents that may be biologically degraded and that do not involve the recycling of any non-environmentally friendly chemicals or solvents, for example, are to be used. The components used are to be particularly admissible for the use in the fields of animal feed, food and pharmaceuticals. By coating an active protein, particularly an enzyme protein, its contact with inhibiting substances, as it is, e.g., present in mineral premixes, is to be prevented too, and storage-stable mixtures are to be produced.

It has now surprisingly been found that by applying one or several layers of a micronized product from leguminous plants to particles that contain a biologically active protein or by coating, covering, encapsulating of kernels that contain a biologically active protein, the thermal stability (thermostability) of these biologically active proteins may be strongly increased without affecting the activity of the thus coated protein in any other way. A coating layer from a micronized product from leguminous plants is thereby applied, whereby only the external layer of the protein-containing particles contacts the micronized product from leguminous plants. This results in an effective protection of the thus coated biologically active proteins against increased temperatures in processing methods that involve thermal loads of the products. The coating also serves as protection of, e.g., enzymes during the storage in mixtures with substances acting as inhibitors. Moreover, the method enables the production of very highly concentrated products (enzyme products) with the properties according to the invention as regards the thermostability in the further steps of manufacture and use.

Therefore, the invention relates to a protein-containing agent or substance with increased thermal stability comprising a core and a coating layer characterized in that the core comprises at least one biologically active protein and that the coating layer comprises a micronized product from leguminous plants. Furthermore, the invention relates to a method for the production of such a protein-containing agent, whereby a suspension that comprises a micronized product from leguminous plants is applied to a core that comprises at least one biologically active protein and the thus obtained coated particles are dried. The invention also relates to the use of a micronized product from leguminous plants to increase the thermal stability of biologically active proteins, preferably in methods for the production of a pelletized product.

The method according to the invention or the use according to the invention has the advantage over the methods of the state of the art that small granulate particles, which optimally protect the enzyme against the denaturing conditions at high temperatures and humidity, may also be formed. For example, particles according to the methods of extrusion (WO 00/47060) or according to the production by plow blade mixers (WO 01/04279) are about 400-2,000 μm. The particles according to the invention may be 100-300 μm, and, thus, they are very suitable to produce homogenous products, particularly if the particles show high enzyme activities.

The method according to the invention or the use according to the invention is universally suitable to increase the thermostability, particularly of enzyme preparations, and is, e.g., applicable to the use in pelletized animal feed; due to the high solubility of the substance the enzyme is also immediately biologically available. The amount of material to be applied is small as opposed to the methods of the state of the art and, thus, enables the production of highly active enzyme particles.

In the method according to the invention for the production of the coated protein-containing agent according to the invention a micronized product from leguminous plants, preferably a micronized soy product, is applied to a core that comprises at least one biological protein. The micronized product from leguminous plants may be applied by simple spraying and subsequent drying. However, the micronized product from leguminous plants may also be directly applied in a step in the respectively selected process such as in a fluidized bed dryer or flat bed spray dryer.

According to the invention, the coating layer comprises or consists of a micronized product from leguminous plants. According to the invention, this concerns micronized legumes (fabaceae) that contain fat, protein, starch and other carbohydrates as storage agents. Examples thereof are micronized products from peas, lentils, beans, peanuts, soy beans, lima beans, garbanzos, cow beans, lupines. Preferably, the micronized product from leguminous plants is a micronized soy product. The micronized product from leguminous plants may be used as such for the coating of biologically active proteins or in combination with additives that are technologically necessary and reasonable. These may be, e.g., acids and bases for adjusting the pH value and/or salts, buffer substances, etc. Micronized components of leguminous fruits may be used, e.g., the residues after defatting. The use of a micronized product from leguminous plants from the respective leguminous full fruit is preferred. The micronized product from leguminous plants may be thereby produced according to any methods for micronization known per se. The micronization is a short-time heating process with high temperature. The starting material is thereby heated with pulses of few seconds without significant water loss. In this connection the micronization by means of infrared pulses having wavelengths of 1.8 to 3.4 μm is particularly suitable.

The micronized product from leguminous plants may be suspended by stirring or homogenized by Ultraturrax, possibly after adjusting a specific pH value, in aqueous solutions. In production processes this is carried out by in-line homogenizers just in front of the spray nozzle to avoid separation of the suspension. The thus obtained suspension may be sprayed on any kind of particles. The particles may be derived from, e.g., spray drying processes, granulation processes, agglomeration processes or extrusion processes. The coating layer may then be applied according to the batch method in separate devices such as fluidized bed dryers, coaters with and without Wurster tube, ProCell devices etc. or also continuously, e.g., in a flat bed spray dryer following the granulation or agglomeration or regarding a ProCell device in one of the back spray chambers before the final drying process. All ways of applying the coating layer require attention to a high final drying of the total product, which is to obtain, e.g., >90% dry mass, preferably >92%, more preferred >95%, the most preferred >97% dry mass. The applied mass of micronized product from leguminous plants may be 2 to 50% (w/w) dry mass based on the used protein-containing particle.

The micronized product from leguminous plants is preferably a micronized soy product and particularly preferably the below-described product “micronized soja” by Micronizing Company (UK) Ltd.

The micronized soy product used to apply a coating layer in the Examples was produced on the basis of fully fat soy beans.

An exemplifying micronized soy product that may be used for the purposes of the present invention is the product “micronized soja” by Micronizing Company (UK) Ltd., Charnwood Mill, Framlingham, Suffolk, IP13 9PT (www.micronizing.com). This product is produced by infrared micronization, whereby infrared light with a wavelength of 1.8 to 2.3 μm is used. A typical manufacturing process for this product comprises the following steps:
The pre-purified products are consistently boiled on a vibration conveyor at 120° C. with infrared, whereby the partial pressure of water steam heavily increases and the contained components such as proteins, cellulose, hemicellulose, etc. are, therefore, digested. Anti-nutritive factors, which are, e.g., present in soy, are also thereby inactivated and the material achieves a very high nutritional value as animal feed.
The heated product is stored in a container for a time between 5 and 15 minutes.
Then the product is grinded to flakes in a high-performance mill and subsequently cooled down to room temperature. As regards soy and other oil-containing seeds and crops, the grinding process results in a digest of the oil bodies and a release of the contained oil.
The flakes are put into a disintegrator to obtain full-fat flour. The particle size of the thus obtained particles for the application is below 1 mm in diameter, better below 0.5 mm, even better below 0.3 mm in diameter.

It is obvious that this method or a corresponding method with the same result may also be used for the micronization of other legumes. Method parameters such as, e.g., temperature and time, as well as the sequence of the method steps may be thereby varied depending on the type and amount of the legume to be micronized. Such variations are in the range of knowledge of a person skilled in the art and may be determined by simple routine experiments. It is essential that a reduction of the particle size to smaller 1 mm in diameter, preferably smaller 0.5 mm in diameter, even more preferred smaller 0.3 mm in diameter is obtained by the micronization, even in combination with homogenisation before the spraying.

The micronized product from leguminous plants is applied in an amount in the range of 50% (w/w) based on the dry mass of the particles containing the biologically active protein and the micronized product from leguminous plants, preferably in the range of 2 to 50%, more preferred in the range of 5 to 35% and most preferred in the range of 7 to 25%. The micronized product from leguminous plants as such may thereby be applied in aqueous solution or homogenized and, thus, in a finer disintegration than in pure suspension. The suspension is preferably produced with water, and before application it may be adjusted to a certain pH value with buffer substances and/or bases or acids, or further substances such as, e.g., salts, may be added to improve the effect of the micronized products form leguminous plants.

According to the invention, the core to which the coating layer consisting of micronized product from leguminous plants is applied comprises at least one biologically active protein, optionally together with technologically necessary and reasonable agents. It may also comprise combinations of several biologically active proteins, optionally together with technologically necessary and reasonable additives. The biologically active protein is preferably thermally labile. The biologically active protein is preferably an enzyme. The core may also only consist of the biologically active protein.

This enzyme is preferably selected from phytases, phosphatases, alphagalactosidases, beta-galactosidases, laccases, phospholipases, endoglucanases, particularly endo-beta-1,4-glucanases, endo-beta-1,3(4)-glucanases, endo-1,2-beta-glucanases and endo-1,3-alpha-glucanases, cellulases, xylosidases, galactanases, particularly arabinogalactan-endo-1,4-beta-galactosidases and arabinogalactan-endo-1,3-beta-galactosidases, pectin-degrading enzymes, particularly pectinases, pectin esterases, pectin lyases, polygalacturonases, arabananases, rhamnogalacturonases, rhamnogalacturonan acetyl esterases, rhamnogalacturonan-alpharhamnosidases, pectate lyases and alpha-galacturonidases, mannanases, beta-mannosidases, mannan acetyl esterases, xylan acetyl esterases, proteases, other xylanases, arabinoxylanases, lipolytic enzymes such as lipases, digalactoside diglyceride esterases and cutinases, and other enzymes such as laccases and transglutaminases. The enzyme is particularly preferably selected from phytases, endoglucanases, xylanases or phosphatases.

The core that comprises at least one biologically active protein may comprise further carrier substances and additives, for example, partially hydrolyzed starch products such as corn starch, wheat starch, etc., protein-containing substances such as, e.g., skimmed milk powder, casein hydrolysates, yeast hydrolysates or autolysates, other vegetable ingredients such as, e.g., cellulose, hemicellulose or lignin-containing components and cell extracts, organic and inorganic salts such as, e.g., calcium propionate, citric acid or salts of citric acid, buffering substances, acids and bases for adjusting the pH value.

By applying a coating layer of micronized product from leguminous plants according to the invention, the thus coated biologically active protein obtains a higher temperature stability (thermostability). By the imparted increased thermostability the biologically active proteins become active under conditions at which usually a partial or complete inactivation of the protein would take place. The thermostability imparted to the proteins enables their further processing under thermal conditions, which would otherwise lead to the inactivation of the biologically active protein, as well as a correspondingly longer storage time or a storage at increased temperature. The coating also prevents the contact of the protein with other agents that might act in an inhibiting way on the encased protein during the further processing or storage.

For the obtained increased thermostability according to the invention it is of particular importance that the core that comprises at least one biologically active protein is completely coated by the coating layer of micronized product from leguminous plants. Such a covering coating of the starting substance containing the biologically active protein is usually obtained by a careful process management in applying the coating. It is conceivable that the micronized product from leguminous plants surrounds particularly completely the starting particles due to the micronized state. The use of the micronized product from leguminous plants within the aforementioned amounts also contributes to a preferably complete coating of the starting material. Based on the mass of the protein-containing agent, the smaller the particle size of the protein-containing agent, the higher the amount of micronized product from leguminous plants to be applied. This is caused by the ratio of volume to surface. Compared to the volume, the surface of smaller particles is larger than of large particles.

Due to the increased thermostability conferred by the coating layer from a micronized product from leguminous plants, the thus pre-treated starting material is particularly suitable for applications in methods for the use of agents with small grain size, particularly for the production of pelletized products. Animal feed in particularly is very often used in a pelletized state. Since increased temperatures regularly occur at pelletizing conditions, the method according to the invention or the use according to the invention provides the opportunity to prepare pelletized animal feed products with maximal obtainment of the biological activity of the respectively used proteins.

The coated protein-containing agents or particles according to the invention may be added to further uses as such or in processed form. For example, they may be incorporated into animal feed, food or pharmaceuticals. Corresponding methods and applications are well known to the person skilled in the art.

The attached figures explain the invention in more detail:

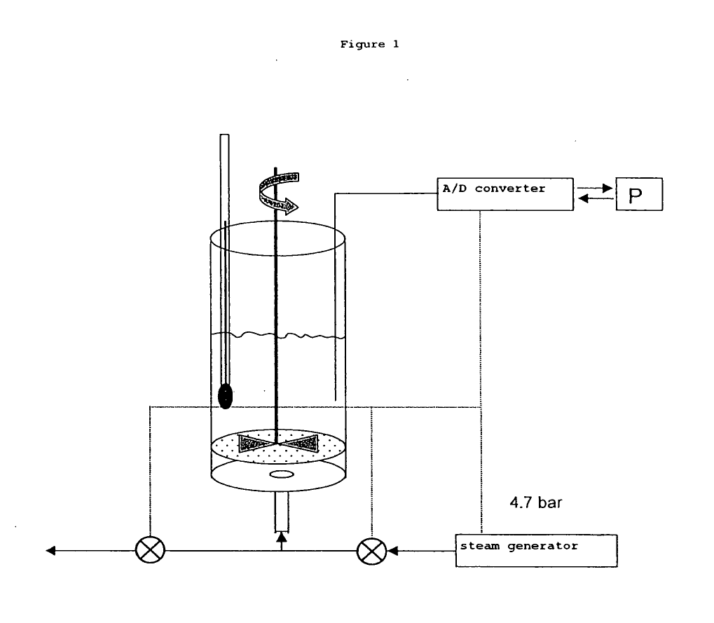

FIG. 1 shows a schematic drawing of a steam generator (streamer; laboratory apparatus):

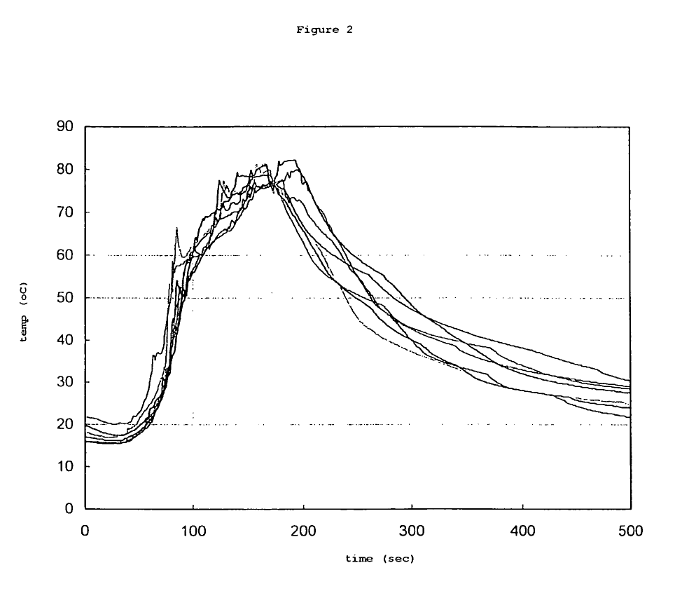

FIG. 2 shows a typical temperature profile of feed/enzyme samples in a steam generator under conditions that correspond to the 80° C. conditions of method A) (see below).

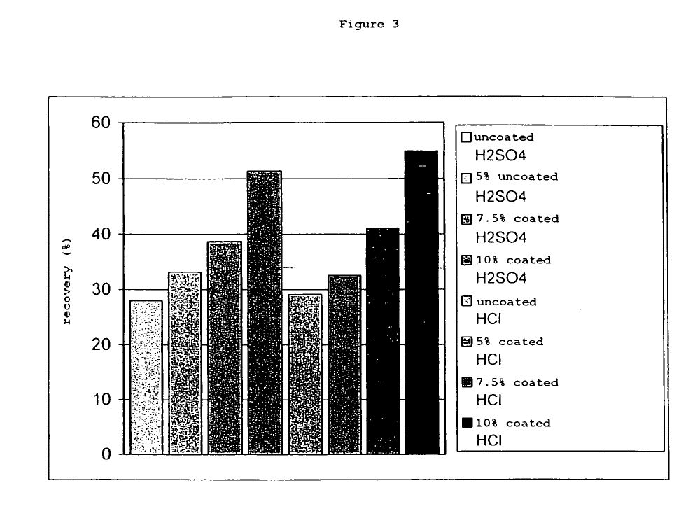

FIG. 3 shows the influence of the coating layer on the thermostability of acid phosphatase after adjustment of the pH value with different acids.

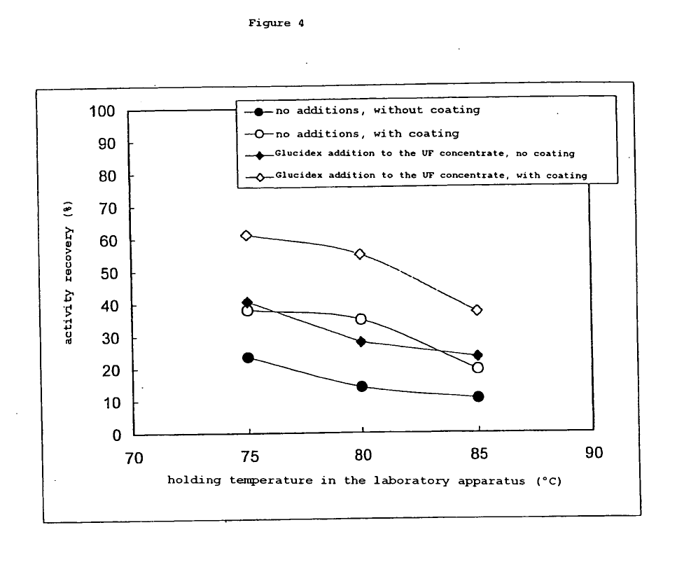

FIG. 4 shows the activity recovery after a thermal load by means of a laboratory streamer at an endoglucanase granulate with and without addition of Glucidex before the application to a Glucidex core and with subsequent coating with 10% (w/w) dry mass of micronized soy.

The invention is explained in more detail on the basis of the below examples:

### EXAMPLES

### Reference Example 1

### Determination of Phytase Activity

The phytase activity is measured in an assay mixture containing 0.5% (w/w) phytic acid (about 5 mM), 200 mM sodium citrate, pH 5.0. After 15 minutes of incubation at 37° C., the reaction is stopped by addition of an equal volume of 15% (v/v) trichloroacetic acid. The released phosphate ions are quantitatively determined by mixing of 100 μl of the assay mixture with 900 μl H₂O and 1 ml 0.6 M H₂SO₄, 2% (w/v) ascorbic acid and 0.5% (w/v) ammonium molybdate after incubation at 50° C. and a duration of 20 min at 820 nm. Standard solutions of potassium phosphate are thereby used as reference.

### Reference Examples 2

### Determination of Acid Phosphatase Activity

The determination of the acid phosphatase activity is carried out analogously to the determination of the phytase in Reference Example 1 with the difference that the substrate solution is 10 mM α-glycerophosphate in 250 mM glycine/HCl buffer, pH 2.5.

### Reference Example 3

### Determination of endo-β-1,4-glucanase Activity

β-glucanase hydrolyzes the glycosidic bindings in dissolved barley glucane. The released reducing sugars react with the DNS reagent to a color complex, the content of which is photometrically determined at 540 nm.

1 unit of the β-glucanase activity is defined as the amount of enzyme that is needed to release 1 μmol of sugar equivalents that reduce glucose per minute under defined conditions. The enzyme reaction is carried out at pH 5.5 and 40° C.

DNS reagent:

10 g NaOH pellets and 200 g sodium tartrate are dissolved in 800 ml de-ionized water. After addition of 10 g 3,5-dinitrosalicylic acid methyl ester (DNS), it is stirred for 1 h at 30-40° C. and filled up to 1 l.

Barley-glucane Substrate Solution:

1 g barley glucane (Megazyme # P-BGBM) is suspended in 60-70 ml de-ionized water and then heated to 80-85° C. while stirring until the glucane is dissolved. Subsequently, it is cooled down to room temperature while stirring. After addition of 10 ml buffer, pH 5.5, it is filled up to 100 ml.

To determine the β-glucanase activity, a batch of 0.9 ml substrate solution and 0.1 ml diluted enzyme solution is incubated for 15 min at 40° C. After addition of 1.5 ml DNS reagent, the batch is heated in a boiling water bath for 20 min and then cooled down to room temperature. After addition of 2 ml de-ionized water, the optical density is determined at 540 nm in a photometer. The measurement is carried out against blanks using water instead of enzyme solution. The determination of the β-glucanase activity is carried out by a calibration curve with glucose.

### Example 1

### Applying the Coating Layer

Two preferred embodiments are described below, according to which a coating layer of a micronized soy product may be applied. The above-mentioned product “micronized soya” of Micronizing Company (UK) is thereby used. These method variants are cited in Examples 3 to 10.

The coating layer may be applied in the following method variants:

Method A): Applying the coating layer in a fluidized bed dryer (Aerocoater) of the type Strea-1, company Aeromatic-Fielder AG, Bubendorf, Switzerland

In this method the coating layer that comprises micronized soy product is applied to a granulate that comprises a biologically active protein by a specific fluidized bed dryer. The granulate is a protein granulate produced in a way known per se.

An air compressor produces dry, fat-free air compressed to 6 bar. Typical charge sizes of protein-containing granulate that may be processed in the fluidized bed dryer of the type Strea-1 may be determined according to standard methods. The coating experiments are carried out in Strea-1 with bottom spray devices (Aerocater). Typical charge sizes are in the range of 50 to 300 g granulate. The tempered compressed air is introduced with a pressure of 0.6 bar by a 1.2 mm spray nozzle, which is disposed at the centre of the air distribution plate at the bottom. The air distribution plate is modified in such a way that there is an air flow >95% into the center, and the remaining 5% completely flow into the marginal areas of the vessel to avoid a contact of the humid granulate with the walls. A short Wurster pipe is used in most of the coating experiments and is attached at 0.8 cm above the air distribution plate. In some experiments it is difficult to maintain the flow of the granulate in the Wurster pipe, and the coating is then carried out in the absence of the pipe. Typical flow rates of the coating liquids comprising the suspended or homogenized micronized soja” are 1 to 6 ml min⁻¹. The suspension used for spraying was an aqueous suspension with a dry mass between 5% and 25% of “micronized soja”, which was homogenized by means of an Ultraturrax shortly before the spraying. To avoid a blocking of the nozzle in an embodiment with a bottom spray device, the air inlet temperature should not exceed 60° C. during the spraying. When the complete “micronized soja” is applied, the air inlet temperature is increased to 80-95° C. to dry the coated granulate to a final water content of 5 to 10%. The thus obtained granulate may be used as such or may be processed.

Method B): Appling the coating layer in a fluidized bed dryer of the type GPCG1, company Glatt Systemtechnik GmbH, Dresden, Germany

In this method the coating layer that comprises a micronized soy product is applied to a granulate that comprises a biologically active protein in a specific fluidized bed dryer. The granulate is a protein granulate produced in way known per se.

Higher amounts of granulates, which are derived from, e.g., an industrial process with a spray granulator, type SBD 76, company APV Anhydro AS, Denmark, are coated in a GPCG1, Glatt, in the top spray method or the bottom spray method. About 500 g protein granulate are put in the GPCG1 in each experiment. The coating conditions are the following:

Top spray coating:

Granulate temperature: 40-43° C.

Air temperature: 55° C.

Bottom spray coating

Granulate temperature: 38-40° C.

Air temperature: 55° C.

The “micronized soja” is stirred to a 10% (w/w) suspension in water and homogenized by an in-line homogenizator before the spraying.

The spraying rate of the homogenized micronized soy suspension is adjusted in such a way that there is no coagulation of the granulate, and, thus, the particle size of the used granulate only changes insignificantly due to the coating. The typical spray rate for the suspension is about 5-15 ml min⁻¹. A two-jet nozzle is used to introduce and apply the 10% (w/w) micronized soy suspension.

The coating is usually 10% (w/w) based on the dry mass of the used granulate; however, it may also be between 5 and 30% (w/w).

### Example 2

### Carrying Out the Thermal Load Tests

The proteins, particularly enzymes, contained in the products coated by “micronized soja” may be tested for their thermostability according to the following methods. The coated products are thereby subjected to a thermal load in one of the methods described below. These methods exemplarily relate to the testing of the thermostability of particles that contain an enzyme. The thermostability of particles that contain other biologically active proteins may be tested analogously.

In the following methods the enzyme activity that is added to the end products to be produced is determined by a measuring method that is specific for the enzyme to be tested before and after the thermal load (see Reference Examples 1-3). The enzyme activity recovered after the thermal load is indicated as percentage of the used enzyme activity in the form of the percental recovery. The thermal loads are carried out to demonstrate and compare the efficacy of the coating layer with uncoated and coated material.

Method A): Testing the thermostability in a pilot pelletizing plant (practicable at the Institute of Biotechnology of the Research Institute for Food and Molecular Biotechnology, Kolding, Denmark; the test may also be carried out in another corresponding pelletizing plant.)

Coated or uncoated granulates that contain the enzymes fungal or bacterial phytase, acid phytase or endoglucanase and are produced according to method A) or B) of Example 1 are premixed with wheat flour of type 550 to obtain 300 g of an enzyme-containing premix. This premix is mixed with 15 kg animal feed at the Institute of Biotechnology of the Research Institute for Food and Molecular Biotechnology, Kolding, to guarantee an optimal dilution for mixing it in 285 kg animal feed and a well-determinable enzyme activity in the pelletized material. The used amount of coated granulate is between 0.1 and 10 g per kg animal feed in most experiments depending on the activity height of the enzyme contained in the coated material and, thus, leads to an enzyme activity of about 5 units (U) g⁻¹ animal feed of phytase or about 6 units (U) g⁻¹ of acid phosphatase or about 230-270 units (U) g⁻¹ of endoglucanase. The 300 kg animal feed that are treated in the pelletizing plant have the composition of Table 1.

TABLE 1
Composition of the animal feed to be pelletized

| Component | [%] |
|---|---|
| Hipro Soja 48 | 20 |
| soy oil | 4.75 |
| vitamin/minerals, Beta Avitren 90 | 0.25 |
| enzyme premix* | 300 g |
| wheat | ad 100 |

*with fungal or bacterial phytase, acid phosphatase or endoglucanase

The pelletizing conditions are as follows:

Horizontal mixer with 700 l volume, 48 rpm; the amount that is mixed is in the range of 80 to 300 kg.

A horizontal screw conveyor is attached to the mixer to empty the mixer, the conveying speed of which is adapted to the following step.

The mixer is Kahl cascade mixer (conditioner) with a length of 130 cm, 30 cm in diameter, 155 rpm and 37 chambers. The throughput is 300 kg per hour. Feed and the enzyme-containing particles according to the invention are in contact with the wet steam for about 30 s. (The conditions as regards steam and temperature are indicated in Table 2).

The steam, which is produced at the Dan Stoker high-pressure boiler, flows through a pressure-regulation valve into the cascade mixer with 2 bar overpressure. The valve controls the amount of steam that flows into the cascade mixer and leads to the heating of the enzyme-containing feed therein.

The conditioned material is pelletized by a Simon Heesen press and subsequently air-cooled in a perforated box flowed with 1,500 m³ h¹. The pelletizing press runs with 500 rpm at an input of 7.5 kW and thereby produces pellets of 3×20 mm.

TABLE 2
Steam conditions to obtain the different temperatures in pelletizing
animal feed that contains the particles according to the invention
in the plant at the Institute of Biotechnology of the Research Institute
for Food and Molecular Biotechnology, Kolding, Denmark

| set-point temperature [C. °] | steam [%] | actual temperature [° C.] |
|---|---|---|
| 70 | 3.6 | 69.8-70.0 |
| 75 | 3.9 | 74.8-74.9 |
| 80 | 4.3 | 79.7-79.9 |
| 85 | 4.6 | 84.7-84.8 |
| 90 | 5.0 | 89.8-90.0 |
| 95 | 5.4 | 94.9-95.1 |

The thermal load for testing the thermostability of coated particles may also be correspondingly conducted in other plants. Subsequently, the still remaining enzyme activity in the coated and uncoated particles in the pelletized animal feed is determined according to Reference Examples 1-3.

Method B): Carrying out the thermal load tests in a laboratory apparatus—Laboratory Streamer

Thermal load tests, in short thermostability tests, that are carried out in closed vessel are at best an indication of an increased storage stability, since they act as an accelerated storage test. However, to test an increased thermostability under the pelletizing conditions for feed, a laboratory method was developed by which the thermostability of enzymes in the incorporation into feed and a load under the conditions occurring in a feed mill (see method A) may be reliably determined. It was found that the major part of the enzyme inactivity occurs when enzyme-containing feed is subjected to an increased temperature and humidity, that is, under conditions that occur during the steam conditioning of feed. To determine this degree of inactivation, an enzyme granulate in animal feed is subjected to a steam heat in a device in laboratory scale. The method is configured for a defined temperature (75-85° C.). A device in which such tests may be carried out is depicted in FIG. 1 and consists of a brass vessel with a perforated insert on which the feed is introduced and also has a mechanism to introduce steam into the feed. Steam is charged from a usual steam generator (Euroflex) with 4.7 bar pressure. The amount of steam that enters the vessel is regulated by the two control valves, which also regulate the removal of water condensate from the part of the vessel that is below the insert with the feed. It is important that the steam pipes are free from condensate and are already at operating temperature before being attached to the brass vessel.

The vessel is charged with the feed to be tested (50-100 g, typically 50 g, composition see Table 1), which contains the enzyme-containing particles according to the invention, and steam is introduced into the vessel. The sample is stirred with a long barbell having a thermoelement at the end or the thermoelement is separately introduced into the animal feed at a probe. The temperature of the feed sample is consistently recorded by means of a personal computer by connecting the probe to a data logger (DrDAQ, company Pico Technology, UK) after A/D conversion of the measuring signal.

When the desired temperature is obtained (usually 80° C. or 85° C.), the temperature is maintained for 30 to 60 seconds, the feed is taken from the vessel and cooled down in a flat plastic container. Typical temperature profiles for these tests at 80° C. are depicted in FIG. 2 (temperature profile of feed/enzyme samples in steam corresponding to the conditions of 80° C. in a method according to method A)). The obtained maximal temperature and the time span that is maintained for this temperature may be regulated by switching the valves and/or regulating the dispensed amount of steam.

The experiments on thermoload were always carried out in the laboratory apparatus as double determinations at least. The areas below the temperature curves were integrated to determine whether repeated samples were subjected to the same total thermal load. The exposure time at the defined temperature of 80° C. or 85° C. is, however, important. The still remaining enzyme activity in the coated or uncoated particles in the mixture is determined according to Reference Examples 1-3.

### Example 3

### Temperature Stability of the Phytase of a Filamentous Fungus

Part A

UF concentrate of Aspergillus niger var. awamori phytase, produced with a recombinant Trichoderma Reesei strain according to, for example, WO 94/03612 was dried in a APV spray ganulator with different additions dissolved in the UF concentrate (cf. Example 1, method B)) and then coated by the method according to Example 1, method B). The granulates that were coated in the top spray method contained 15% (w/w) citric acid based on the dry mass of the UF concentrate, and the pH value was adjusted to pH 3. The granulate that was coated by the bottom spray method moreover contained 10% (w/w) maltodextrin, which was dissolved in the UF concentrate before drying. All UF concentrates that were adjusted in this way were injected in the APV dryer with spray pressures of 80 to 180 bar. The particle sizes resulting therefrom were between 200 and 300 μm for all granulates produced. Thermal load tests (in short thermostability tests) were carried out according to Example 2, method A). The results are shown in Table 3.

TABLE 3
Increase of the heat stability of Aspergillus phytase in
granulates by coating with 10% (w/w) “micronized soja”.

| | recovery enzyme activity [%] | | |
|---|---|---|---|
| | | coated by | coated by |
| temperature [C. °] | uncoated | top spray | bottom spray |
| 70 | 46.7 | 99.3 | 84.7 |
| 75 | 31.9 | 70.4 | 71.5 |
| 80 | 21.0 | 43.3 | 32.1 |
| 85 | 9.5 | 28.1 | 18.3 |
| 90 | 5.3 | 14.1 | 10.9 |
| 95 | 3.6 | 5.0 | 5.1 |

To adjust the pH value of the UF concentrate before the spray granulation, citric acid was used.

The particle size of all uncoated and coated products was between 200 and 300 μm, measured by means of laser diffraction on a Malvern device. The results in Table 3 show that an increase in the thermostability of the enzyme enclosed in the coated particles under the thermal loads that are present in the pelletizing process is obtained independently from the selected additives in the UF filtrate. Moreover, the spray pressure, which also influences the size and form of the developing spray granulates (particle with enzyme activity), has no influence on the improvement of the thermostability of the enzyme enclosed in the coated spray granulate to be observed in the pelletizing process. Particles (granulates) according to the invention, in which at thermal load the enclosed enzyme undergoes a lower inactivation than in the untreated particle, may be produced by the top spray method as well as the bottom spray method.

Part B

The UF concentrate of Part A was dried to spray granulate without core in a ProCell5, Glatt, by addition of 50% (w/w) skimmed milk powder (30% lactose) based on the dry mass of the UF concentrate and after adjustment of the pH value with sodium citrate to pH 5.1. This takes place by the spraying of the mentioned solution with a dry mass of about 15-20% in the top spray method against the airflow in the ProCell5 by a two-jet nozzle with a spraying rate of 6-25 g min⁻¹ exceeding the batch process. The temperature of the additional air is 70°-95° C. at an amount of additional air of 90-100 m³ h⁻¹ during the spraying, whereby the product temperature is maintained at 50° C. When the entire solution has been sprayed, the temperature of the additional air is increased to max. 100° C. for further 5 min, whereby the product temperature does not exceed 55° C. to obtain the desired amount of dry mass of >95% in the obtained granulate. In a batch method about 2 l of the mentioned solution are processed to spray granulate. The particle size of the spray granulates was below 300 μm. The thus obtained spray granulates were coated with a 10% (w/w) homogenized suspension of “micronized soja” by means of the method described in Example 1 A), whereby the coating had an amount of 10% (w/w) based on the dry mass of the spray granulates. Thermal load tests (thermostability tests) according to Example 2, method A) were carried out. The results of the activity recovery are shown in Table 4.

TABLE 4
Increase of the heat stability of Aspergillus phytase
in granulates by coating with 10% (w/w) “micronized soja”,
whereby skimmed milk powder was dissolved in the UF concentrate
before the processing to spray granulates.

recovery enzyme activity [%]

| temperature [C. °] | uncoated | coated |
|---|---|---|
| 70 | 70.1 | 77.8 |
| 75 | 45.4 | 61.0 |
| 80 | 29.4 | 42.8 |
| 85 | 9.1 | 13.0 |
| 90 | 0.4 | 7.2 |
| 95 | 0.2 | 0.4 |

The results in Table 4 show that other additions but citric acid to the Aspergillus phytase in the drying may also act advantageously on the enzyme stability, in particular the addition of skimmed milk powder. However, a further improvement of the thermostability of the encased enzymes was not only reached in part A but also in part B by the application of a coating from “micronized soja” independent of the composition of the enzyme-containing spray granulate to be coated.

### Example 4

### Effect of the Coating Degree of Micronized Soy on the Thermostability of the Phytase of a Filamentous Fungus

A 10% suspension of “micronized soja” was produced in water, as it was also used for coating in Example 3. The insoluble components were separated by centrifugation. The thus developed water-soluble fraction was used for an experiment to examine if the effect of the increase in the thermostability of the encased phytase in the thermoload tests is caused by the phytic acid, the substrate of the phytase, contained in the “micronized soja”. The aliquot amount, as it is necessary in the coating to obtain a 10% w/w coating with “micronized soja”, was used. The sales product FINASE® PC of the company ROAL OY, which is produced from a UF concentrate according to Example 3, is used.

FINASE® PC was coated by the method according to Example 1, method A). The thermal load was carried out according to the method of Example 2, method B) at 80° C. for 30 seconds, and subsequently the residual enzyme activity in the treated material was determined.

Table 5 shows the efficacy of aqueous extract from “micronized soja” on the thermostability of the enzyme contained in the particle during the thermal load test.

TABLE 5

| | | activity recovery |
|---|---|---|
| no. | particle description | [%] |
| 1 | FINASE ® Pc | 46 |
| 2 | basis 300 g FINASE ® Pc, spraying with the | 46 |
| | aqueous extract of a 10% suspension of “mi- | |
| | cronized soja” | |
| | suspension that corresponds to a 5% coating | |
| | amount with “micronized soja” | |

As can be understand from a comparison of samples 1 and 2, a coating with the aqueous extract from soy has no stabilizing effect on the enzyme in the spray granulate during the pelletizing process. Soy flour is rich in phytic acid, the substrate of the phytase. Phytic acid is said to have a stabilizing effect on phytase (WO 98/55599). The present experiment shows that the increase in the thermostability obtained in Example 3, as it could be shown under the thermoload of the pelletizing process, cannot be attributed to this effect. An infiltration of phytic acid into the marginal layers of the granulate, as it could take place while coating, is, therefore, not sufficient to explain the observed increase in the thermostability under the conditions of the pelletizing process either.

### Example 5

### Coating of Bacterial (E. coli) Phytase with “Micronized Soja” in Spray Granulates with Core of Glucidex

In the present example a bacterial (E. coli) phytase-containing spray granulate with core of Glucidex is coated with micronized soy.

Granulation

800 g Glucidex 12 (corn starch hydrosylate of the company Roquette, France, containing 1% glucose, 2% disaccharide and 97% higher polysaccharides) were presented in STREA 1, Aeromatic Fielder, and sprayed with 500 g UF concentrate of E. coli phytase (pH 2.5, adjusted with sulfuric acid), produced by a recombinant T. reesei strain (WO 2006/042719).

Coating

The spray granulates were then coated according to the method of Example 1, method A) (5% (w/w) “micronized soja” based on dry masses), and the thermostability of the phytase in the spray granulates was tested according to the method of Example 2, method A). The results are shown in Table 6.

TABLE 6
Increase in the thermostability of E. coli phytase in particles from spray
granulation with Glucidex 12-core by coating with “micronized soja”

| | activity recovery [%] | activity recovery [%] |
|---|---|---|
| temperature [° C.] | uncoated | coated 5% |
| 70 | 61.7 | 94.2 |
| 75 | 45.5 | 73.3 |
| 80 | 36.9 | 50.4 |
| 85 | 21.8 | 23.7 |
| 90 | 6.0 | 10.1 |
| 95 | 0.2 | 1.3 |

The data in Table 6 shows that the thermostability of the E. coli phytase in the enzyme-containing spray granulate under the test conditions of Example 2, method A) was already increased by the application of a coating layer from “micronized soja” of 5% (w/w) based on the dry mass of the spray granulate.

### Example 6

### Coating of a Spray Granulate without Core, Containing a Bacterial (E. coli) Phytase

The present example shows the increase in the thermostability of bacterial phytase in a spray granulate without core by the application of a coating with “micronized soja”.

Granulation

In a further experiment 2 kg UF concentrate of E. coli phytase (see Example 5) in a ProCell 5, Glatt, was used to produce spray granulates without core (see Example 3, part B). 30% (w/w) skimmed milk powder (30% lactose content) based on the total protein of the UF concentrate and 4.4 g Ca propionate were added to the UF concentrate. The pH value was adjusted to a value of pH 5 by correction by means of sulfuric acid or sodium hydroxide solution.

Coating

The spray granulates were subsequently coated according to the method of Example 1, method A) (10% (w/w) “micronized soja” based on dry masses), and the thermoload was carried out according to the method of Example 2, method A), and, thus, the thermostability of the phytase in the spray granulates was tested. The results are shown in Table 7.

TABLE 7
Thermostability of E. coli phytase in
spray granulates without Glucidex core

| | activity recovery [%] | activity recovery [%] |
|---|---|---|
| temperature [° C.] | uncoated | coated 10% |
| 70 | 85.7 | 99.9 |
| 75 | 49.6 | 82.1 |
| 80 | 19.3 | 41.6 |
| 85 | 4.5 | 18.3 |
| 90 | 0.3 | 9.7 |
| 95 | 0.3 | 4.7 |

As the results show, there is an increased thermostability for the phytase contained in these spray granulates coated by “micronized soja” under the conditions that are present in the pelletizing experiments.

Comparing the data for the uncoated and the coated spray granulates of Example 5 with 6 shows that a Glucidex core is not responsible for the increase in the thermostability, as it is obtained by the application of the coating with “micronized soja”. The activity recoveries for the non-coated particles of Examples 5 and 6 show a different distribution of the thermostability of the enzyme under the selected experimental conditions; however, the results are on a similar level and each time far below the results that were obtained by the coating with “micronized soja”. It was not possible to demonstrate an influence of the core, as it is, for example, postulated in WO 98/54980 and WO 00/24877. WO 98/54980 particularly attributes a stabilizing effect on the obtained enzyme in the thermoload to the absorption of the enzyme solution in a carbohydrate polymer and subsequent drying, as they occur at the pelletizing conditions in animal feed. This stabilizing effect could not be obtained in the present case of spraying, i.e., not absorbing, an enzyme solution on a carbohydrate polymer.

The results in this example also show that the effect is not limited to specific kinds of phytases, although phytases were used in Examples 3 and 6. In Example 3 a phytase of a filamentous fungus and in Example 6 a phytase of bacterial origin were used, which, however, only show a homology of 18% on amino acid level and are, thus, to be regarded as two completely independent proteins.

### Example 7

### Coating of a Granulate Containing an Acid Phosphatase of a Filamentous Fungus

In the present example the increase in the thermostability of the acid phosphatase of a filamentous fungus was examined by a coating of the granulate with “micronized soja”.

Granulation

200 ml UF concentrate of Aspergillus niger pH 2.5 acid phosphatase, produced by a recombinant T. reesei strain, e.g., according to WO 94/03612, was spray-dried on 500 g Glucidex 12 in STREA 1, Aeromatic Fielder, after adjustment of the pH value to pH 4 with HCl or H₂SO₄. The amount of CaSO₄ dropped out in the adjustment of the pH value with sulfuric acid was separated by centrifugation before the spray granulation.

Coating

The spray granulates were coated with different amounts of “micronized soja” according to the method of Example 1, method A). The thermoload of the acid phosphatase in the spray granulates was carried out according to the method described in Example 2, method B) at 85° C. for 60 s, and the thermostability was determined by measurement of the residual activity being present afterwards. The results are shown in FIG. 3.

The results shown in FIG. 3 show that the obtained increase in the thermostability of the acid phosphatase in the coated spray granulates under the conditions of the pelletizing process according to Example 2, method B) are directly connected with the amount of applied micronized soy.

Moreover, it was possible to show by the selection of the acid that the amount of free calcium in the UF concentrate being spray-granulated had no influence on the thermostability of the enzyme as opposed to phytase, an enzyme closely related to the acid phosphatase, such as shown in WO 97/05245, U.S. Pat. No. 5,972,669, U.S. Pat. No. 5,827,709 as well as EP 0 758 018, where the addition of bivalent cations such as calcium, magnesium or zinc, particularly as addition in the form of inorganic salts, to the UF concentrate during the spray granulation led to an increase in the thermostability of the phytase under certain thermoload tests. By the use of sulfuric acid in the pH adjustment, there are precipitations of calcium sulfate and, thus, a decrease in the calcium concentration compared to the UF concentrates in which the pH adjustment was carried out by hydrochloric acid.

### Example 8

### Effect of Glucidex or Skimmed Milk Powder on the Thermostability of Granulate Containing Acid Phosphatase with and without Coating with Micronized Soy

In the present example the influence of the addition of Glucidex and skimmed milk powder to the UF concentrate in the spray granulation of the enzyme compared to the effect of a coating layer of micronized soy on the thermostability of a coated spray granulate of acid phosphatase was tested.

Granulation

UF concentrate of Aspergillus niger pH 2.5 acid phosphatase, produced by a recombinant T. reesei strain (see Example 6), was spray-granulated to particles in the size of up to 300 μm in a ProCell5, Glatt, with different additions that were dissolved in the UF concentrate (30% Glucidex or 50% skimmed milk powder [SMP]) (see Example 3, part B). The pH value of the solutions used for the spray granulation was pH 4 each.

Coating

The coating was carried out according to Example 1, method A). The applied amount of “micronized soja” was 10% (w/w) based on the dry mass. The results are shown in Table 8.

TABLE 8
Thermostability of uncoated and coated spray granulates of acid
phosphatase

| | activity recovery [%] | | | activity recovery [%] | | |
|---|---|---|---|---|---|---|
| | without coating | | | with coating | | |
| temperature | no | 30% | 50% | no | 30% | 50% |
| [° C.] | addition | Glucidex | SMP | addition | Glucidex | SMP |
| 70 | 83.5 | 91.7 | 100.0 | n.d. | 93.8 | 100.0 |
| 75 | 76.5 | 87.8 | 96.0 | n.d. | 99.9 | 100.0 |
| 80 | 50.0 | 82.0 | 93.4 | n.d. | 95.9 | 96.6 |
| 85 | 25.1 | 57.4 | 64.8 | n.d. | 76.6 | 95.4 |
| 90 | 2.0 | 7.4 | 3.5 | n.d. | 13.6 | 13.9 |

n.d.: not defined

The comparison of the data of Table 8 shows that the addition of Glucidex or skimmed milk powder to the UF concentrate of the acid phosphatase before the spray granulation also have a stabilizing influence on the acid phosphatase of Aspergillus niger var. awamori in the pelletizing experiments. The addition of skimmed milk powder to the UF concentrate of the enzyme before the spray granulation proved to be positive not only in the E. Coli phytase but also in the acid phosphatase and the phytase from Aspergillus niger var. awamori as regards the thermostability of the enzymes under the conditions in the pelletizing experiment. However, in all cases the thermostability (expressed as % of activity recovery) of the enzyme in the spray granulates could be further improved by the application of the coating layer of “micronized soja” in the pelletizing experiment. Independent of the used construction or composition of the spray granulate containing the enzyme, the application of “micronized soja” results in an increase in the thermostability of the enzyme in the entire particle.

### Example 9

### Thermostability of an Endo-1,4-β-Glucanase granulate with Glucidex Core with and without Coating with “Micronized Soja”

In the present example the thermostability of the enzyme endo-1,4-β-glucanase I from Trichoderma reesei was determined in a granulate with a Glucidex core after coating with “micronized soja”. This enzyme shows two different enzyme activities—endo-1,4-β-glucanase and an equally high endo-β-1,4-xylanase activity.

Granulation

UF concentrate of Trichoderma reesei endoglucanase 1 (endo-1,4-β-glucanase), produced by a recombinant T. reesei strain (Karhunen et al., Mol Gen Genet 1993, 241:515-522), was used to produce different spray granulates in a STREA 1, Aeromatic Fielder. The spray granulates were obtained by spray drying of 200 ml UF concentrate on 500 g Glucidex 12 core material, Roquette, France, or by spray drying of 200 ml UF concentrate in which 10% (w/w) Glucidex 12 based on the dry mass were additionally dissolved before the spray drying on the Glucidex core.

Coating

The granulates obtained by the spray drying were coated according to the method of Example 1, method A) by application of a 10% suspension of “micronized soja”. The amount of the coating material was 10% (w/w) based on the dry mass of the coated granulate.

The increase in the thermostability of the endoglucanase contained in the granulates was determined according to the method that was described in Example 2, method B).

FIG. 4 shows the activity recovery of endoglucanase I in the granulates with and without application of a 10% coating layer on “micronized soja” after carrying out the thermal load in a laboratory apparatus (Laborstreamer). In the thermal load experiments the obtained thermostability of endoglucanase I from Trichoderma reesei is also dependent of the addition of maltodextrin to the UF concentrate before the spray drying. FIG. 4 shows that independent of the used construction of the enzyme-containing granulate or the addition to the UF concentrate in the production of the enzyme-containing granulate, the application of “micronized soja” in a coating method leads to an increase in the thermostability of the enzyme contained in the coated granulate under the experimental conditions. Furthermore, the improvement of the thermostability that is obtained by the application of the coating layer is larger than the one that may be obtained by the addition of 10% Glucidex 12 to the UF concentrate in the spray drying.

### Example 10

### Thermostability of endo-1,4-β-glucanase with and without Coating with “Micronized Soja”

In the present example the theremostability of endo-1,4-β-glucanase (endoglucanase 1) in a spray granulate without Glucidex core was examined after coating with “micronized soja”.

Granulation

In the UF concentrate of Trichoderma reesei endoglucanase 1, produced by a recombinant T. reesei strain (Karhunen et al., Mol Gen Genet 1993, 241:515-522), 20% Glucidex 12 (w/w) based on the dry mass was dissolved and the pH value was adjusted to pH 4.5 by H₂SO₄/NaOH. This concentrate was dried in a ProCell5, Glaft, to spray granulates in the size of up to 300 μm (see Example 3, part B).

Coating

The spray granulates were coated according to Example 1, method A). The applied layer of “micronized soja” was 10% (w/w) based on the dry mass. The results of the activity recovery after the thermal load according to Example 1, method A) are shown in Table 9.

TABLE 9
Thermostability of enoglucanase I from Trichoderma reesei with and
without coating in granulates that were produced without Glucidex core

| | activity recovery [%] | activity recovery [%] |
|---|---|---|
| temperature [° C.] | without coating | with coating |
| 70 | 83.5 | 94.2 |
| 75 | 68.0 | 81.2 |
| 80 | 41.5 | 49.4 |
| 85 | 24.9 | 30.9 |
| 90 | 5.7 | 13.3 |

The data in Table 9 shows that the thermostability of endoglucanase I from Trichoderma reesei, which is enclosed in the granulate, may also be improved in the absence of a core of Glucidex by coating with “micronized soja”. A core of a carbohydrate material is no precondition for the increase in the thermostability by a coating with “micronized soja”.

1. An agent with increased thermal stability comprising a core and a coating layer characterized in that the core comprises at least one biologically active protein and that the coating layer comprises a micronized product from leguminous plants.

2. The agent according to claim 1 characterized in that the micronized product from leguminous plants is a micronized soy product.

3. The agent according to claim 1 characterized in that the core further comprises maltodextrin.

4. The agent according to claim 1 characterized in that the core further comprises skimmed milk powder.

5. The agent according to claim 1 characterized in that the biologically active protein is an enzyme.

6. The agent according to claim 5 characterized in that the enzyme is selected from phytases, endoglucanases, xylanases or phosphatases.

7. The agent according to claim 1 characterized in that the coating layer is 2-50% (w/w) based on the total dry mass of the substance.

8. The agent according to claim 7 characterized in that the coating layer is 2-30% (w/w).

9. The agent according to claim 8 characterized in that the coating layer is 5-25% (w/w).

10. An animal feed composition characterized in that it comprises or consists of an agent according claim 1.

11. A food composition characterized in that it comprises or consists of an agent according to claim 1.

12. A pharmaceutical composition characterized in that it comprises or consists of an agent according to claim 1.

13. A method to produce an agent according to claim 1 characterized in that a solution that comprises a micronized product from leguminous plants is applied to a core that comprises at least one biologically active protein and the thus obtained coated particles are dried.

14. The method according to claim 13 characterized in that the application and drying of the solution that comprises a micronized product from leguminous plants is carried out in a fluidized bed dryer.

15. The method according to claim 13 characterized in that the micronized product from leguminous plants is a micronized soy product.

16. A use of a micronized product from leguminous plants to increase the thermal stability of biologically active proteins in methods for the production of a granulated product.

17. A use of a micronized product from leguminous plants to increase the thermal stability of biologically active proteins in methods for the production of animal feed.

18. A use of a micronized product from leguminous plants to increase the thermal stability of biologically active proteins in methods for the production of a food.

19. A use of a micronized product from leguminous plants to increase the thermal stability of biologically active proteins in methods for the production of a pharmaceutical composition.

20. The use according to claim 16 characterized in that the micronized product from leguminous plants is a micronized soy product.
